(19) United States
(12) Patent Application Publication
(10) Pub. No.: US 2013/0000627 A1
(43) Pub. Date: Jan. 3, 2013

(54) HEATABLE GARMENT

(76) Inventor: Harvey T. Bart, New York, NY (US)

(21) Appl. No.: 13/600,993

(22) Filed: Aug. 31, 2012

Related U.S. Application Data
(63) Continuation of application No. 12/249,500, filed on Oct. 10, 2008.

(51) Int. Cl. A61F 7/08; A41D 1/04; B32B 37/14; A41D 1/00

(52) U.S. Cl. 126/204; 2/69; 2/102; 156/60

(57) ABSTRACT

A heatable garment includes a front and back panel having an opening there between for receiving a wearer's head. The front and back panels include a first and second layer of material. A plurality of sealed enclosures is formed between the first and second layers. At least one of the plurality of enclosures is disposed on the front panel and the back panels. The plurality of enclosures each includes a heating device having a source of energy for producing heat. The enclosures are sealed to retain the heating devices therein.

The present application is a continuation of U.S. application Ser. No. 12/249,500, filed Oct. 10, 2008, now pending, the entire contents of which are incorporated by reference herein.

### FIELD OF THE INVENTION

The present invention relates to a garment, and more particularly a garment having heat-generating devices.

### BACKGROUND OF THE INVENTION

Often times in outdoor cold environments it is desirable to have garments which not only insulate a wearer from the cold but also develop heat independent of the wearer. Garments such as gloves, socks and coats have been made including devices which generate heat in order to keep their wearers comfortable. Traditionally, such garments use electric heating coils connected to a portable electrical power supply, e.g., batteries. Accordingly, the garment does not work by simply insolating the wearer from the ambient conditions to maintain natural body heat, but also has an independent heating element to provide additional warmth. The effectiveness of such garments, however, relies on the power supply being available and active when the need for the warm garment arises. Such garments are not suitable for long term storage since the batteries tend to discharge over time.

Pocket warmers using fuel, such as lighter fluid or naphtha, to create a catalytic reaction are also known in the art and are placed within pockets existing in a garment in order to keep a wearer warm. Such warmers need to be refueled, and it can be difficult to both start and maintain the reaction.

Heating devices relying on slow and sustained chemical reactions in order to generate heat are known in the art. Such devices include exothermic packs which are activated upon exposure to the atmosphere. The exposure to the oxygen in the atmosphere creates a chemical reaction with the contents of the packs generating heat for a certain amount of time. Once the chemical reaction runs its course, the heat is no longer generated. Such exothermic heating packs are disclosed, for example, in U.S. Pat. No. 5,342,412.

The disposable air-activated heating packs may be placed within pockets formed in garments. For example, such a garment is shown in U.S. Pat. No. 5,302,806 which is directed to a heated vest having pouches for removably holding an air activating heating pack. The pouches include an opening which permit a user to insert the pack and remove the pack therefrom once it is spent. The garment is formed of traditional clothing type material.

In the designs of the prior art, a user must have both the specialized garment and heating packs available in order to use the garment in situations where additional heating is required. However, it is typical that when an individual requires the use of the heated garment, the heating packs or batteries are not available; and therefore, the garment can only be worn as a standard garment without having any additional heating. This is unacceptable especially when the garment is to be used for emergency purposes where a user is relying on the additional heating in order to provide heating in extreme cold or severe weather conditions.

Accordingly, it would be desirable to provide a garment which has heating devices integrally formed therewith such that the garment is ready to be worn and used as a heating device when desired.

### SUMMARY OF THE INVENTION

According to aspects illustrated herein, there is provided a heatable garment that includes a front and back panel having an opening there between for receiving a wearer's head. The front and back panels include a first and second layer of material. A plurality of sealed enclosures is formed between the first and second layers. At least one of the plurality of enclosures is disposed on the front panel and the back panels. The plurality of enclosures each includes a heating device having a source of energy for producing heat. The enclosures are sealed to retain the heating devices therein.

According to other aspects illustrated herein, there is provided a single use heatable vest including a front panel and a back panel placeable on a wearer to cover a portion of a wearer's torso. The front and back panels define an opening for receiving a wearer's head. The front and back panels are formed of a first layer and second layer of supple material. A plurality of sealed enclosures is formed between the first and second layers. The plurality of enclosures includes an air-activated heating device non-removably secured within the enclosure. A securement device secures the front panel to the back panel.

According to still other aspects illustrated herein, there is provided a disposable, personal heating system including a garment having a front and a back panel having an opening there between for receiving a wearer's head. The front and back panels include a first and second layer of material. A plurality of enclosures is formed between the first and second layers. At least one of the plurality of enclosures is disposed on the front panel and on the back panel. The plurality of enclosures includes an exothermic heating device disposed within the enclosures. An environmental barrier surrounds the garment, and removal of the barrier activates the heating devices.

According to further aspects illustrated herein, there is provided a method of warming a mammal including:

obtaining a garment including a front and a back panel having an opening there between for receiving a wearer's head;

the front and back panels including a first and second layer of material;

a plurality of enclosures formed between the first and second layers, and at least one of the plurality of enclosures being disposed on the front panel and on the back panels;

the plurality of enclosures each including an exothermic heating device disposed within the enclosures; and

an environmental protective barrier surrounding the garment, wherein removal of the barrier activates the heating devices;

opening the protective barrier thereby activating the heating devices; and

placing the garment on a wearer.

According to still further aspects illustrated herein, there is provided a method of making a single use heating garment including:

forming a first layer of material to a predetermined shape including an aperture therein sized to receive a wearer's head;

forming a second layer of material to a predetermined shape;

overlying the first layer with the second layer;

forming an array of enclosures between the first and second layers,

placing an exothermic heating device in each enclosure;

bonding the first and second layer together to seal the enclosures and retain the heating devices therein.

### BRIEF DESCRIPTION OF THE DRAWINGS

FIG. 1 is a perspective view of a garment of the present invention.

FIG. 2 is a top plan view of the garment of FIG. 1.

FIG. 3 is a cross-sectional view of the garment taken along line 3-3 of FIG. 2.

FIG. 3A is a cross-sectional view of an alternative embodiment of the garment of FIG. 3.

FIG. 4 is a perspective view of a system of the present invention.

FIG. 5 is an alternative embodiment of a garment of the present invention.

### DETAILED DESCRIPTION OF THE PREFERRED EMBODIMENTS

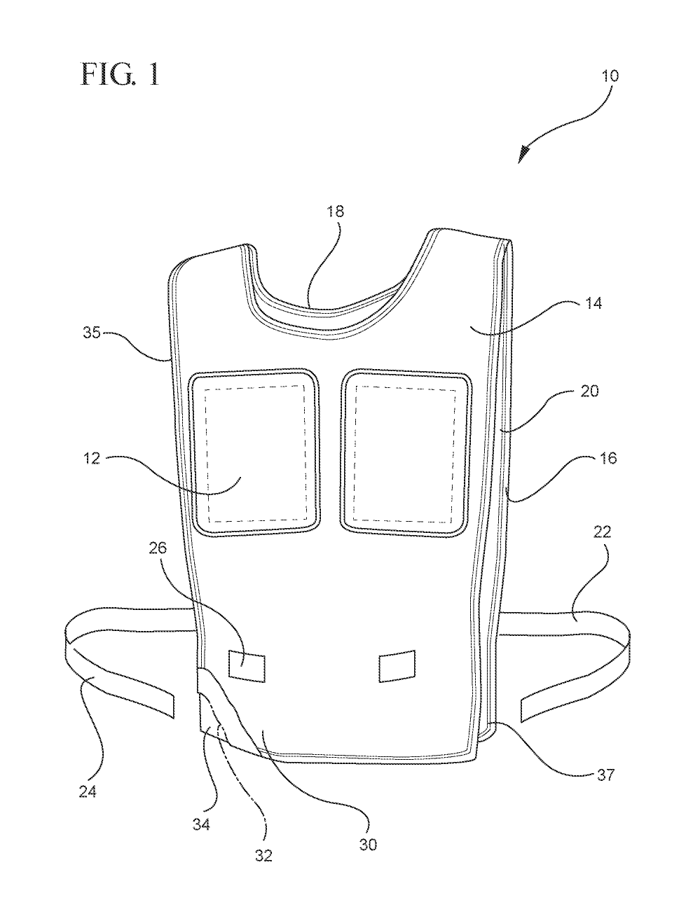

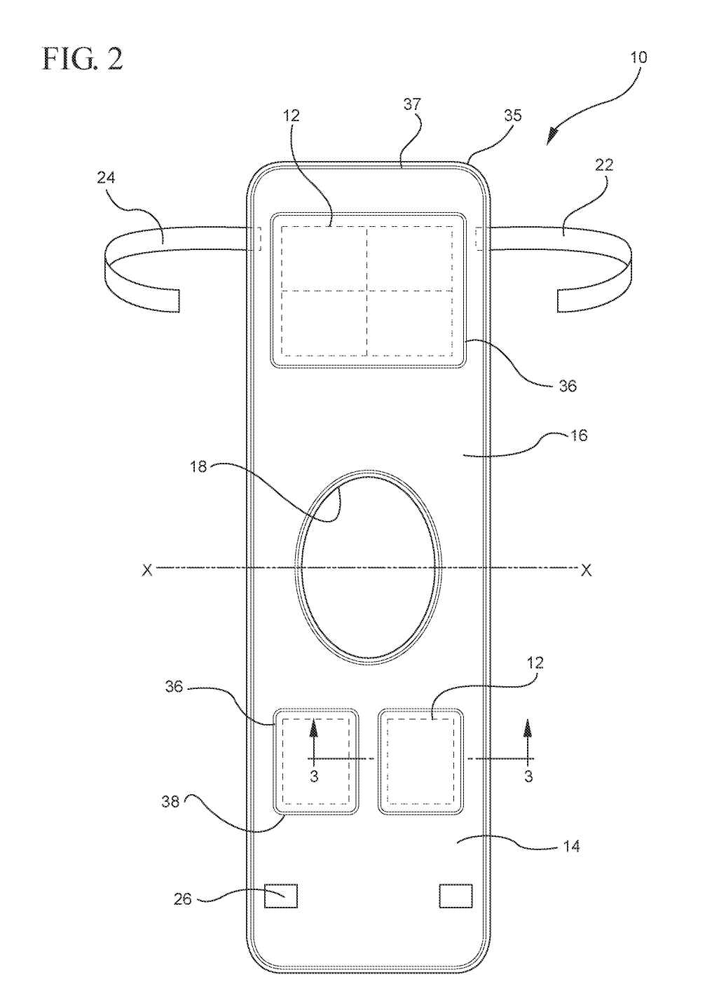

With reference to FIGS. 1 and 2, the present invention provides a garment 10 including one or more heating devices 12 disposed thereon. The garment 10 may be in the form of a vest including a front panel 14 and a back panel 16. An opening 18 disposed between the front 14 and rear 16 panels permits a wearer to place their head therethrough. The front panel 14 is foldable over the back panel 16 generally around an axis X-X extending across at least part of the head opening 18. The garment 10 covers the torso of the wearer. Spaces 20 formed between the side edges of the front and rear panels permit a wearer to place their arms therethrough. The garment 10 may further include a securement device 22 including a pair of straps 24 which are attached to and extend from the back panel over the front panel and may be secured by a fastening device 26. The connection between the straps 24 and fastening device 26 may effected by a hook and loop fastening, snaps, hooks, buckles, or any other type of connecting mechanisms known in the art. The securement device 22 may be adjustable to accommodate the size of the wearer.

The heating devices 12 may be disposed on both the front 14 and back 16 panels of the garment 10. In a preferred embodiment, a plurality of heating devices 12 are disposed on each of the front and back panels. Therefore, a wearer is surrounded by heating devices 12 when the garment 10 is worn.

The heating devices 12 may include devices which generate heat and which may have incorporated therewith the source of energy for producing the heat. In a preferred embodiment, heating devices 12 may include an air-activated heating device wherein exposure to the air causes an exothermic reaction generating heat. Air-activated heating devices are well known in the art. Such devices typically include components such as iron oxide, water and salt. In a preferred embodiment heating device 12 may include a packet comprised of a face side-nylon non-woven fabric inside-polyethylene laminate film pierced needle holes. Disposed within the packet is iron powder (50-60%), water (15-25%), activated charcoal (5-10%), water vermiculite (1-3%), salt (2-5%), wood powder (2-5%) and water absorptive powder (0.1-1%). When exposed to the oxygen in the air, the iron oxidizes resulting in a generation of heat. The period of heat generation may be approximately 6 to 8 hours, for example. The heating device 10 may a single-use type device wherein once it is activated and the reaction is completed, it may be disposed of. Heating devices 12 may be formed as individual packets, or alternatively formed as a plurality of packets connected together.

Alternatively, the heating devices 12 may include other types of heat generating devices that may be either chemically or electrically powered.

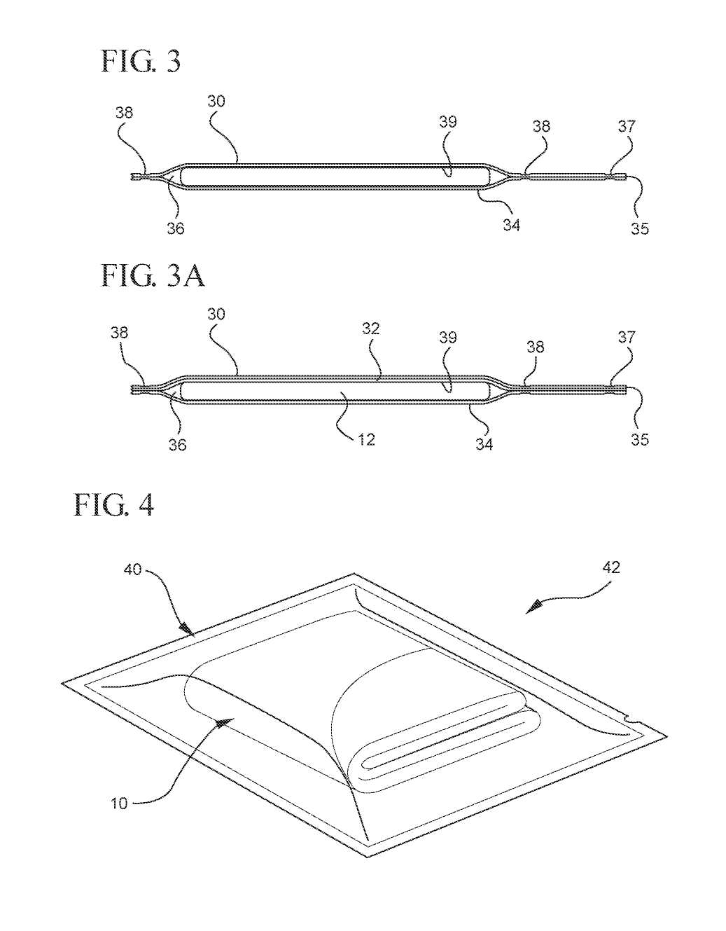

With further reference to FIG. 3, the garment 10 may be formed from multiple layers of supple material secured together. An outer layer 30 which faces away from a wearer's body may include a non-woven fabric which is trimmed to the proper size. The fabric may include a nylon spundbond material consisting of 98% polycapronamide and 2% titanium dioxide. It is within the contemplation of the present invention that other materials including woven materials may be used for the various layers. An inner layer 34 which faces and lies against the wearer may be formed of a material the same or similar to the outer layer, namely, a non-woven material.

In an alternative embodiment, an insulating intermediate layer 32 may be disposed between the inner and outer layers. Layer 32 may be formed of a foam type material. Layer 32 may have an insulating property greater than that of the inner 34 and outer 30 layers.

The various layers which form the garment 10 may be cut from a roll of material such that the layers have a similar profile. The layers may then be stacked up with the inner layer 34 disposed over the outer layer 30. The edges 35 of the layers may be secured together by an edge seam 37 to form the garment 10. The securement of the layers may be achieved, for example, by heat welding the edges together such as by radio frequency or ultrasonic welding, or by use of an adhesive, or by stitching the layers. The straps 24 have ends which are attached to the back panel 14 by inserting a portion of the strap between the inner 34 and outer 30 layers and extending the edge seam 37 so that it captures the strap end.

With reference to FIG. 3, in the preferred embodiment, the heating devices 12 are sandwiched between the inner layer 34 and the outer layer 30. The heating devices 12 are preferably non-removably secured to the garment. Accordingly, the garment 10, along with the heating devices 12 which have the energy to produce heat, form a unitary structure. The heating devices 12 may be sealed between the inner layer 34 and outer layer 30 in an enclosure 36. A plurality of enclosures 36 may be formed on the garment 10 to accommodate a plurality of heating devices 12. Enclosure 36 may be sized to receive an individual heating device such as shown on the front panel 14, or may sized to include a plurality of connected heating devices 12 such as the enclosure 36 on the back panel 16. Accordingly, the size of the enclosures may be determined by the size and number of heating devices 12 to be contained therein. The heating packs 12 may be placed between the inner 34 and outer 30 layers in the desired locations. To form the enclosures 36, which retain one or more of the heating devices 12, a sealed seam 38 is made completely surrounding the heating device 12. Seams 38 may be formed in a manner similar to that used to forming seams 37, for example, by radio frequency welding. Accordingly, the heating devices 12 which include the energy source for producing the heat are permanently sealed within the garment 10 and cannot be separated therefrom. An adhesive layer 39 may be provided between the heating devices 12 and the outer layer 30 to maintain the heating device's position within the enclosure 36.

In an alternative embodiment, shown in FIG. 3A, the heating devices 12 are sandwiched between the inner layer 34 and the insulating intermediate layer 32. Accordingly, the heat generated by the heating devices 12 is maintained between the insulating intermediate layer 32 and the wearer. The adhesive layer 39 may be between the heating devices 12 and the intermediate layer 32. In this embodiment, the heating devices 12 are retained in the enclosure 36 by a surrounding seam 38.

With reference to FIG. 4, the garment 10 may be folded and inserted within an environmental protective barrier 40 which surrounds the garment 10 forming a ready-use package 42. This barrier 40 may include a bag formed of a material which does not permit air and especially oxygen to pass therethrough. In a preferred embodiment, the barrier may be formed of a polypropylene film of vinylidene chloride-coated material which has low vapor and gas permeability. The barrier 40 may be evacuated and hermetically sealed such that the garment 10 and the heating devices 12 are isolated from the air. In this state, the heating devices 12 are unused and not activated. The garment 10 may remain in this ready state until needed by a wearer.

When a wearer desires to use the garment 10, they may open the protective barrier 40 and remove the garment 10 therefrom. By exposing the garment 10 to air, the heating devices 12 are activated and begin to generate heat. The garment 10 may be placed on the wearer by placing over the head to provide heat for a finite period of time. It is within the contemplation of the present invention that the garment may be of a type worn by humans; however, the garment could also be configured to be placed on animals where extra warmth is desired.

Once the heating devices 12 are spent and are no longer generating heat, the wearer may remove the garment and dispose of it. Another package 42 may be maintained by the wearer in case the need again arises. Upon opening the package 42, the user is present with both the garment and a fresh set of heating devices 12 every time. There is no need to separately obtain a garment and the unused heating devices. This can be especially helpful in emergency situations where the need for additional warmth is in unexpected. A package may be carried such as in the trunk of a car or in emergency services equipment, and only opened when the need arises.

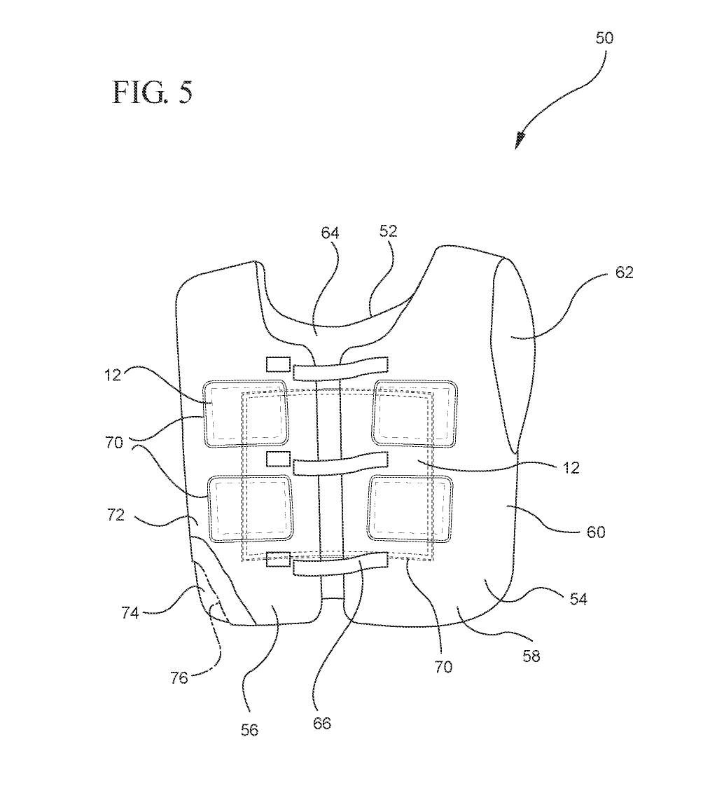

With further reference to FIG. 5, an alternative embodiment of a garment 50 is shown. The garment 50 includes a jacket-type vest having a back panel 52 and a split front panel 54 formed of two flaps 56 and 58. A transition 60 between the back panel 52 and the split front panel 54 includes openings 62 for receiving a wearer's arms. A gap 64 is also formed between the split front panel 54 and the back panel 52 in order to allow the user's head to extend therethrough. Fastening devices 66 including straps, or other type devices may be located on the front panel 54 of the garment and permit the two flaps 56 and 58 of the front panel 54 to be brought together and closed around the wearer. The heating devices 12 may be disposed on and sealed within enclosures 70 on the two flaps of the front panel 54 and the back panel 52. Enclosures 70 may be formed in a similar manner as enclosures 36 described above. The garment 50 may be formed of multiple layers including an outer layer 72 an inner layer 74 in a manner similar to garment 10 described above. Alternatively, an intermediate layer 76 of foam material, such as insulating layer 32 described above, may be included. Garment 50 may be stored in a hermetically sealed in a environmental protective barrier to prevent activation of the heating devices 12 until the need arises.

While the garment shown herein is represented as a vest, it is within the contemplation of the present invention that the garment may take the form of other wearable items such as jackets, shirts, pants, gloves, hats socks, etc.

It will be appreciated that various of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A heatable garment comprising:
a front and back panel having an opening there between for receiving a wearer's head;
the front and back panels including a first and second layer of material;
a plurality of sealed enclosures formed between the first and second layers, at least one of the plurality of enclosures being disposed on the front panel and the back panels; and
the plurality of enclosures each including at least one heating device having a source of energy for producing heat; the enclosures being sealed to retain the heating devices therein.

2. The garment as defined in claim 1, wherein the first layer forms an outer layer and the second layer forms an inner layer adapted to face toward a wearer.

3. The garment as defined in claim 2, further including an intermediate layer disposed between the inner and outer layers.

4. The garment as defined in claim 3, wherein the intermediate layer is an insulating layer.

5. The garment as defined in claim 1, wherein a plurality of heating devices are positioned in a single enclosure.

6. The garment as defined in claim 1, wherein the heating devices are air-activated exothermic devices.

7. The garment as defined in claim 1, wherein a seam is formed around each of the plurality of enclosures to prevent removal of the heating devices therefrom.

8. The garment as defined in claim 1, wherein the first and second layers are formed of a non-woven material.

9. The garment as defined in claim 1, wherein the front and back panel form a vest adapted to cover a wearer's torso.

10. The garment as defined in claim 9, wherein the front panel includes a pair of flaps extending from the back panel.

11. The garment as defined in claim 9, wherein the front panel is foldable over the back panel around an axis extending through the opening.

12. A single use heatable vest comprising:
a front panel and a back panel placable on a wearer to cover a portion of a wearer's torso;
the front and back panels defining an opening for receiving a wearer's head;
the front and back panels being formed of a first layer and second layer of supple material;
a plurality of sealed enclosures formed between the first and second layers;
the plurality of enclosures including an air-activated heating device non-removably secured within the enclosure; and
a securement device for securing the front panel to the back panel.

13. The vest as defined in claim 12, wherein at least one of the first and second layers of material permit the transmission of oxygen.

14. The vest as defined in claim 12, wherein the heating devices are surrounded by a seam.

15. The vest as defined in claim 12, wherein an intermediate insulating layer is disposed between the first and second layers.

16. The vest as defined in claim 12, wherein the front panel includes a pair of heating devices and the back panel includes four heating devices.

17. A disposable personal heating system comprising:
a garment including a front and back panel having an opening there between for receiving a wearer's head;
the front and back panels including a first and second layer of material;
a plurality of enclosures formed between the first and second layers, and at least one of the plurality of enclosures being disposed on the front panel and the back panel;
the plurality of enclosures including an exothermic heating device disposed within the enclosures; and
an environmental barrier surrounding the garment, wherein removal of the barrier activates the heating devices.

18. The system as defined in claim 17, wherein the environmental barrier retards the transmission of oxygen.

19. The system as defined in claim 18, wherein the environmental barrier includes a bag which is hermetically sealed.

20. The system as defined in claim 17, wherein the garment includes a vest and the front and back panels are adapted to surround a wearer's torso.

21. The system as defined in claim 17, wherein the first and second layers are formed of nonwoven material.

22. The system as defined in claim 17, wherein the heating devices are non-removably secured within the enclosures.

23. A method of warming a mammal comprising:
obtaining a garment including a front and back panel having an opening there between for receiving a wearer's head;
the front and back panels including a first and second layer of material;
a plurality of enclosures formed between the first and second layers, and at least one of the plurality of enclosures being disposed on the front panel and the back panels;
the plurality of enclosures each including an exothermic heating device disposed within the enclosures; and
an environmental protective barrier surrounding the garment, wherein removal of the barrier activates the heating devices;
opening the protective barrier thereby activating the heating devices; and
placing the garment on a wearer.

24. The method as defined in claim 23, including removing the garment after the heating elements have stopped producing heat.

25. The method as defined in claim 24, including disposing of the garment after the heating elements have been spent.

26. A method of making a single use heating garment comprising:
forming a first layer of material to a predetermined shape including an aperture therein sized to receive a wearer's head;
forming a second layer of material to a predetermined shape;
overlying the first layer with the second layer;
forming an array of enclosures between the first and second layers,
placing an exothermic heating device in each enclosure;
bonding the first and second layer together to seal the enclosures and retain the heating devices therein.

27. The method as defined in claim 26, wherein bonding the first and second layer together to seal the enclosures includes welding the first and second layer.

28. The method as defined in claim 26, including disposing an insulating layer between the first and second layer and disposing the heating devices between the insulating layer and the second layer.

29. The method as defined in claim 28, wherein the first and second layers are formed of the same material, and the insulating layer is formed of a different material having an insulating property greater then that of the first and second layers.
